(19) United States
(12) Patent Application Publication
(10) Pub. No.: US 2014/0357602 A1
(43) Pub. Date: Dec. 4, 2014

(54) Combination Therapies For Treating Metabolic Disorders

(71) Applicant: Genmedica Therapeutics SL, Barcelona (ES)

(72) Inventors: Eric Mayoux, Eschau (FR); Luc Marti Clauzel, Barcelona (ES); Silvia Garcia Vicente, Sant Feliu de Llobregat (ES); Marta Serrano Munoz, Barcelona (ES); Antonio Zorzano Olarte, Barcelona (ES); Alec Mian, Barcelona (ES)

(21) Appl. No.: 13/829,499

(22) Filed: Mar. 14, 2013

Related U.S. Application Data
(63) Continuation of application No. 12/724,756, filed on Mar. 16, 2010, now abandoned.
(60) Provisional application No. 61/160,610, filed on Mar. 16, 2009.

(51) Int. Cl. A61K 31/198; A61K 45/06; A61K 31/60

(52) U.S. Cl. CPC A61K 31/198; A61K 31/60; A61K 45/06
USPC 514/162

(57) ABSTRACT

This invention is directed to pharmaceutical combinations comprising an antioxidant agent, an anti-inflammatory agent, and optionally at least one other anti-diabetic agent useful for treating metabolic disorders. This invention also encompasses pharmaceutically acceptable compositions comprising an antioxidant agent, an anti-inflammatory agent, optionally at least one other anti-diabetic agent, and at least one pharmaceutically acceptable carrier. The combinations and compositions of this invention are useful as methods for treating metabolic disorders including diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease in a mammal, particularly a diabetic mammal, and specifically a human patient. This invention is particularly directed to pharmaceutical compositions comprising an lipoic acid, one or more anti-inflammatories selected from the group consisting of diflunisal, diclofenac, dexibuprofen, dexketoprofen, naproxen, and salicylate, and optionally one or more pharmaceutically acceptable carriers. The compositions of this invention are useful as methods for treating metabolic disorders including type II diabetes, insulin resistance, beta-cell dysfunction, and hyperglycemia in a patient, particularly a diabetic patient.

### CROSS-REFERENCE TO RELATED APPLICATIONS

This application is a continuation of U.S. patent application Ser. No. 12/724,756, filed Mar. 16, 2010, which is hereby incorporated herein by reference in its entirety, and which claims the benefit of U.S. Provisional Application No. 61/160,610 filed Mar. 16, 2009.

### BACKGROUND

Type II diabetes and its underlying obesity, also called diabesity, is rapidly becoming a worldwide epidemic. There are currently more than 194 million people with diabetes worldwide, and Type II diabetes accounts for up to 90% of diabetics in overall patient populations. It is a well known in the art that diabetes is a risk factor for cardiovascular diseases associated also with dyslipidemia and hypertension. With such long-term complications, diabetes is already the fifth leading cause of morbidity and mortality, imposing a high financial burden on health care costs for society. With a projected doubling of the number of global cases of diabetes by 2030, the development of effective diabetes prevention and treatment strategies is of paramount importance.

Type II diabetes mellitus (T2DM) is a metabolic disorder in which carbohydrate and lipid metabolism are improperly regulated by insulin (insulin resistance) resulting in elevated fasting and postprandial serum glucose levels (hyperglycemia) and increased levels of circulating free fatty acids (FFA) and triglycerides (TG). T2DM is preceded by a long period of insulin resistance during which blood glucose is maintained near normal levels by compensatory hyperinsulinemia. When pancreatic β-cells are no longer able to compensate for insulin resistance by adequately increasing insulin production, impaired glucose tolerance appears. This condition is characterised by an excessive blood glucose concentration in the postprandial phase whereas fasting glucose remains in the normal range. The combination of persistent overfeeding with a sedentary lifestyle leads to overt diabetes characterised by hyperglycemia.

Recently, it has been suggested that oxidative stress and inflammation are key features of obesity and type II diabetes, exacerbating its progression and cardiovascular complications. For example, the antioxidant enzymes responsible for scavenging free radicals have been reported to be diminished in diabetic patients. Glutathione pools become depleted in diabetic patients following frequent and severe hyperglycemic episodes. In particular, pancreatic β-cells that are sensitive to oxidative free radicals become damaged. It is well recognized that pancreatic β-cell dysfunction resulting from prolonged exposure to high glucose and/or elevated free fatty acid (FFA) levels contributes to glucose intolerance and subsequent occurrence of type II diabetes in patients.

Lifestyle modifications, in terms of reduced caloric intake and increased physical activity, can reduce the incidence of type II diabetes up to 58% in the insulin resistant patient population. However, failure of long-term adherence to these modifications limits the potential of this approach. Pharmacological therapies to prevent type II diabetes are an important therapeutic strategy for patients unable to maintain these necessary lifestyle modifications. However, no single anti-diabetic agent can currently be recommended for preventing diabetes. An important distinction to be made here is whether known anti-diabetic agents prevent or delay the onset of diabetes, since the average time period between the onset of β-cell dysfunction and development of diabetes is ten years. This point is illustrated by the fact that several drugs from different classes are on the market today and yet the diabetes population is still growing.

Anti-inflammatory and antioxidant agents may possess potential anti-diabetic properties. Salicylates and aspirin lower glucose levels in patients with diabetes, inducing sometimes hypoglycemic episodes in patients already under anti-diabetic treatments. However, such effects are only reported to be observed when the salicylate dosage is high and associated with undesirable side-effects. Recently, researchers at the Joslin Diabetes Center (Boston USA) reported that treatment of type II diabetes patients with 4 grams/day of salsalate, a non-steroidal anti-inflammatory drug (NSAID) similar to aspirin, lowered fasting glucose and reduced inflammation. Such high doses of NSAID required for chronic treatment of diabetes are known to cause stomach ulceration, bleeding and to have other deleterious effects. These drawbacks effectively preclude the use of anti-inflammatories such as NSAIDs for use as antidiabetic agents.

With regard to antioxidants, research has shown that antioxidant drugs can be used to protect against oxidative stress in experimental models of Type I and Type II diabetes. For instance, nicotinamide, desferrioxamine and N-acetylcysteine have been reported to partially protect islets from immune destruction during low-dose streptozotocin-induced insulitis, a process in which hydroxyl radicals play an important role. However, there has been no demonstration that antioxidant therapy is sufficient as a treatment for T2DM, nor is there any evidence that antioxidants have any specific effects in protecting islet cells other than in experimentally-induced diabetes models that are known to use oxidative stress to produce hyperglycemia.

The need for new drugs able to prevent β-cell failure and disease progression remains especially high in pre-diabetic and type II diabetic patients to slow down or stop the ongoing epidemic. Thus, there remains a need in the art for pharmaceutical compositions that are useful for treating metabolic disorders, particularly including type II diabetes.

### SUMMARY OF THE INVENTION

This invention relates to pharmaceutical combinations comprising certain combinations of an anti-inflammatory agent and an antioxidant agent. Pharmaceutical combinations of this invention are useful for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease in a mammal, particularly a diabetic mammal, and specifically a human patient. Such pharmaceutical combinations are also useful for reducing advanced glycated end products (AGEs), reactive oxygen species (ROS), lipid peroxidation, tissue and/or plasma TNFα and IL6 levels, and for delaying or preventing cardiovascular complications associated with atherosclerosis in a diabetic mammal, particularly a diabetic mammal, and specifically a human patient. Also, pharmaceutical combinations of this invention are useful for protecting pancreatic β-cells, preventing their impairment or failure and subsequent lower insulin secretion in a mammal, particularly a diabetic mammal and specifically a human patient.

As provided herein, this invention is exemplified by the use of pharmaceutical combinations comprising an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen for treating the disorders disclosed herein in a mammal, particularly a diabetic mammal, and specifically a human patient. Particularly-advantageous embodiments of the combinations of this invention are combinations of the antioxidants N-acetylcysteine, alpha-lipoic acid (particularly (R)-alpha-lipoic acid) or taurine with the anti-inflammatories sulindac, salicylic acid, diflunisal, 2-hydroxy-4-trifluoromethylbenzoic acid (HTB), naproxen, paracetamol, diclofenac, dexibuprofen or dexketoprofen. In particular, this invention is exemplified by the use of the pharmaceutical combination comprising N-acetylcysteine (NAC), alpha-lipoic acid or a pharmaceutically acceptable salt thereof or taurine, or a pharmaceutically acceptable salt thereof and an anti-inflammatory for treating the disorders disclosed herein in a mammal, particularly a diabetic mammal and specifically a human patient. Particular examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB; NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal.

Pharmaceutical combinations of this invention, comprising an antioxidant and an anti-inflammatory agent, advantageously show additive or synergistic effects relative to treatment with an antioxidant agent alone or an anti-inflammatory agent alone. Such additive or synergistic effects permit lower dosages of antioxidant and anti-inflammatory agents to be administered while improving the anti-diabetic effect and reducing side effects associated with monotherapy. As provided herein, this invention is exemplified by the use of pharmaceutical combinations comprising an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen for treating the disorders disclosed herein in a mammal, particularly a diabetic mammal and specifically a human patient. Particularly-advantageous embodiments of the combinations of this invention are combinations of the antioxidants N-acetylcysteine, alpha-lipoic acid (particularly (R)-alpha-lipoic acid) or taurine with anti-inflammatories sulindac, salicylic acid, diflunisal, 2-hydroxy-4-trifluoromethylbenzoic acid (HTB), naproxen, paracetamol, diclofenac, dexibuprofen or dexketoprofen. In particular, treatment with the pharmaceutical combination of N-acetylcysteine, alpha-lipoic acid or a pharmaceutically acceptable salt thereof or taurine, and anti-inflammatory compounds including sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen, improves anti-diabetic effects while lowering the risk of gastric bleeding, tinnitus or other deleterious side effects associated with anti-inflammatory administration. Particular examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB; NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal.

This invention thus provides methods for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising an anti-inflammatory agent, an antioxidant agent. In accordance with this invention, methods are also provided for reducing AGEs, ROS, lipid peroxidation, tissue and/or plasma TNFα and IL6 levels, and for delaying or preventing cardiovascular complications associated with atherosclerosis in a mammal, particularly a diabetic mammal, and specifically a human patient that comprise administering to the for treating the disorders disclosed herein in a mammal, particularly a diabetic mammal and specifically a human patient, a therapeutically effective amount, particularly a synergistically effective amount of a pharmaceutical composition of a pharmaceutical combination comprising an anti-inflammatory agent, an antioxidant agent. As provided herein, the methods of this invention for treating diabetes comprise the step of administering a therapeutically-effective amount of a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen for treating the disorders disclosed herein in a mammal, particularly a diabetic mammal and specifically a human patient. Particularly-advantageous embodiments of the combinations of this invention are combinations of the antioxidants N-acetylcysteine, alpha-lipoic acid (particularly (R)-alpha-lipoic acid) or taurine with anti-inflammatories sulindac, salicylic acid, diflunisal, 2-hydroxy-4-trifluoromethylbenzoic acid (HTB), naproxen, paracetamol, diclofenac, dexibuprofen or dexketoprofen. Particular examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal.

The invention also provides pharmaceutically acceptable compositions comprising an anti-inflammatory agent, an antioxidant agent, and at least one pharmaceutically acceptable carrier. The pharmaceutically acceptable compositions of this invention are useful for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease, in a for treating the disorders disclosed herein in a mammal, particularly a diabetic mammal and specifically a human patient. The pharmaceutically acceptable compositions are also useful for reducing AGEs, ROS, lipid peroxidation, tissue and plasma TNFα and IL6 levels, and for delaying or preventing cardiovascular complications associated with atherosclerosis in a for treating the disorders disclosed herein in a mammal, particularly a diabetic mammal and specifically a human patient. As provided herein, the pharmaceutical compositions for treating diabetes comprise a combination of an antioxidant selected from resveratrol, silibinin, alpha-lipoic acid or a pharmaceutically acceptable salt thereof, pterostilbene, N-acetyl cysteine, taurine, idebenone, probucol and curcumin in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient. Particularly-advantageous embodiments of the combinations of this invention are combinations of the antioxidants N-acetylcysteine, alpha-lipoic acid (particularly (R)-alpha-lipoic acid) or taurine with anti-inflammatories sulindac, salicylic acid, diflunisal, 2-hydroxy-4-trifluoromethylbenzoic acid (HTB), naproxen, paracetamol, diclofenac, dexibuprofen or dexketoprofen. Particular examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal.

In another aspect, this invention provides uses for pharmaceutical combinations comprising an antioxidant agent, an anti-inflammatory agent, for preparing, or for the manufacture of, a medicament for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease in a mammal, particularly a diabetic mammal, and specifically a human patient. This invention also provides uses for pharmaceutical combinations comprising an antioxidant agent, an anti-inflammatory agent, and optionally at least one other anti-diabetic agent, for preparing, or for the manufacture of, a medicament for reducing AGEs, ROS, lipid peroxidation, tissue and/or plasma TNFα and IL6 levels, and for delaying or preventing cardiovascular complications associated with atherosclerosis in a mammal, particularly a diabetic mammal and specifically a human patient. As provided herein, medicaments for treating diabetes comprise a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient. Particularly-advantageous embodiments of the combinations of this invention are combinations of the antioxidants N-acetylcysteine, alpha-lipoic acid (particularly (R)-alpha-lipoic acid) or taurine with anti-inflammatories sulindac, salicylic acid, diflunisal, 2-hydroxy-4-trifluoromethylbenzoic acid (HTB), naproxen, paracetamol, diclofenac, dexibuprofen or dexketoprofen. Particular examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal.

In another aspect, this invention provides uses for pharmaceutically acceptable compositions comprising an anti-inflammatory agent, an antioxidant agent and at least one pharmaceutically acceptable carrier for preparing, or for the manufacture of, a medicament for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease in a mammal, particularly a diabetic mammal, and specifically a human patient. This invention also provides uses for pharmaceutically acceptable compositions comprising an anti-inflammatory agent, an antioxidant agent, and at least one pharmaceutically acceptable carrier for the preparation, or manufacture of, a medicament for reducing AGEs, ROS, lipid peroxidation, tissue and/or plasma TNFα and IL6 levels, and for delaying or preventing cardiovascular complications associated with atherosclerosis in a mammal, particularly a diabetic mammal and specifically a human patient. As provided herein, the pharmaceutical compositions for treating diabetes comprise a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient. Particularly-advantageous embodiments of the combinations of this invention are combinations of the antioxidants N-acetylcysteine, alpha-lipoic acid (particularly (R)-alpha-lipoic acid) or taurine with anti-inflammatories sulindac, salicylic acid, diflunisal, 2-hydroxy-4-trifluoromethylbenzoic acid (HTB), naproxen, paracetamol, diclofenac, dexibuprofen or dexketoprofen. Particular examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal.

Also provided by the invention are combinations, pharmaceutical compositions, medicaments, and methods of use thereof, comprising advantageous and effecting compositions comprising at least one antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with one anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient. Combinations comprising advantageous pluralities of antioxidants and anti-inflammatory agents fall within the scope of this invention, particularly wherein such combinations show advantages in efficacy, half-life, absorption, solubility, formulation compatability, stability, or synergistic or complemetary effects. The invention also provides embodiments of the combinations as set forth herein optionally comprising an additional antidiabetes drug.

Specific embodiments of this invention will become evident from the following more detailed description of certain preferred embodiments and the claims.

### BRIEF DESCRIPTION OF THE DRAWINGS

FIG. 1 is a graphical illustration of the combination of (R) lipoic acid and one of diclofenac, dexibuprofen, or dexketoprofen at protecting pancreatic beta cells. INS-1E β-cells were pretreated overnight with (R) lipoic acid 100 μM, diclofenac 100 μM (Diclo), dexibuprofen 500 μM (Dexi), and dexketoprofen 500 μM (Dexket) alone or in combination as indicated in graphics A, B, and C, The effect on the combination is shown.

FIG. 2 is a graphical illustration of the combination of salicylate and (R) lipoic acid at protecting pancreatic beta cells.

FIG. 3 is a graphical illustration of salicylate alone (0.38, 0.75, and 1.5 mmol/kg) and N-acetylcysteine (NAC) alone (0.38 and 0.75 mmol/kg) at preventing increase of glycemia (hyperglycemia) and reduction of plasma insulin induced by Alloxan-mediated β-cell destruction.

FIG. 4 is a graphical illustration of the combination of salicylate (0.38 mmol/kg) and N-acetylcysteine (NAC) (0.19 mmol/kg) at preventing increase of glycemia (hyperglycemia) induced by Alloxan-mediated β-cell destruction.

FIG. 5 is a graphical illustration of the combination of salicylate (0.75 mmol/kg) and N-acetylcysteine (NAC) (0.19 mmol/kg) at preventing increase of glycemia (hyperglycemia) induced by Alloxan-mediated β-cell destruction.

FIG. 6 is a graphical illustration of the combination of salicylate (0.75 mmol/kg) and N-acetylcysteine (NAC) (0.38 mmol/kg) at preventing increase of glycemia (hyperglycemia) induced by Alloxan-mediated β-cell destruction.

FIG. 7 is a graphical illustration of the combination of salicylate (75 mg/kg/day s.c. infusion) and N-acetylcysteine (0.1% drinking water) at improving fasting glycemia of ob/ob mice after 4 weeks of treatment.

FIG. 8 is a graphical illustration of salicylate alone (0.75 mmol/kg/day i.p.), N-acetylcysteine (NAC) alone (0.75 mmols/kg/day i.p.), and the combination of salicylate (0.75 mmols/kg/day) and NAC (0.75 mmols/kg/day) at reducing Free Fatty Acids and Triglycerides in ob/ob mice after 4 weeks of treatment.

FIG. 9 is a graphical illustration of the combination of salicylate (75 mg/kg/day s.c. infusion) and (R) lipoic acid (10 mgs/kg/day i.p.) at improving fasting glycemia and glycosylated haemoglobin (HbA1c) of ob/ob mice after 4 weeks of treatment.

FIG. 10 is a graphical illustration of the combination of salicylate (75 mg/kg/day) and taurine (2.5% drinking water) at improving fasting glycemia of ob/ob mice after 4 weeks of treatment.

### DETAILED DESCRIPTION

This invention provides pharmaceutical combinations comprising an antioxidant agent and an anti-inflammatory agent useful for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease in a mammal, particularly a diabetic mammal, and specifically a human patient. The pharmaceutical combinations comprising an antioxidant agent and an anti-inflammatory agent are also useful for reducing AGEs, ROS, lipid peroxidation, tissue and plasma TNFα and IL6 levels, and for delaying or preventing cardiovascular complications associated with atherosclerosis in a mammal, particularly a diabetic mammal and specifically a human patient. Also, the pharmaceutical combinations comprising an antioxidant agent and an anti-inflammatory agent are useful for protecting pancreatic β-cells, preventing their impairment or failure and subsequent lower insulin secretion in a mammal, particularly a diabetic mammal, and specifically a human patient. Specific, non-limiting examples of pharmaceutical combinations according to the invention are set forth below.

As provided herein, the pharmaceutical compositions for treating diabetes comprise a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient. Particularly-advantageous embodiments of the combinations of this invention are combinations of the antioxidants N-acetylcysteine, alpha-lipoic acid (particularly (R)-alpha-lipoic acid) or taurine with anti-inflammatories sulindac, salicylic acid, diflunisal, 2-hydroxy-4-trifluoromethylbenzoic acid (HTB), naproxen, paracetamol, diclofenac, dexibuprofen or dexketoprofen. The invention particularly provides pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

This invention in certain embodiments provides pharmaceutical combinations comprising N-acetylcysteine, alpha-lipoic acid or a pharmaceutically acceptable salt thereof or taurine, or a pharmaceutically acceptable salt thereof and an anti-inflammatory compound including but not limited to non-steroidal anti-inflammatory drugs (NSAIDs) or a pharmaceutically acceptable salt thereof useful for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease in a mammal, particularly a diabetic mammal, and specifically a human patient. The pharmaceutical combinations comprising N-acetylcysteine, alpha-lipoic acid or a pharmaceutically acceptable salt thereof or taurine, or a pharmaceutically acceptable salt thereof and an anti-inflammatory compound including but not limited to non-steroidal anti-inflammatory drugs (NSAIDs) or a pharmaceutically acceptable salt thereof are also useful for reducing AGEs, ROS, lipid peroxidation, tissue and plasma TNFα and IL6 levels, and for delaying or preventing cardiovascular complications associated with atherosclerosis in a mammal, particularly a diabetic mammal, and specifically a human patient. Also, pharmaceutical combinations comprising N-acetylcysteine, alpha-lipoic acid or a pharmaceutically acceptable salt thereof or taurine, or a pharmaceutically acceptable salt thereof and an anti-inflammatory compound including but not limited to non-steroidal anti-inflammatory drugs (NSAIDs) or a pharmaceutically acceptable salt thereof are useful for protecting pancreatic β-cells, preventing their impairment or failure and subsequent lower insulin secretion in a mammal, particularly a diabetic mammal, and specifically a human patient.

The invention specifically provides such combinations of N-acetylcysteine, alpha-lipoic acid or a pharmaceutically acceptable salt thereof or taurine, with an anti-inflammatory compound including sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen. Particularly-advantageous embodiments of the combinations of this invention are combinations of the antioxidants N-acetylcysteine, alpha-lipoic acid (particularly (R)-alpha-lipoic acid) or taurine with anti-inflammatories sulindac, salicylic acid, diflunisal, 2-hydroxy-4-trifluoromethylbenzoic acid (HTB), naproxen, paracetamol, diclofenac, dexibuprofen or dexketoprofen. Particular examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. Each of these combinations can optionally comprise one or more pharmaceutically acceptable carriers, diluents or excipients. The invention particularly provides pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

As set forth herein, certain combinations of antioxidant and anti-inflammatory agents are useful got treating diabetes in a mammal, particularly a diabetic mammal and specifically a human patient. Specific embodiments of such pharmaceutical combinations provided by the invention include pharmaceutical combinations comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal. Each of these combinations can optionally comprise one or more pharmaceutically acceptable carriers, diluents or excipients. The invention particularly provides pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

Said combinations are useful for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease in a mammal, particularly a diabetic mammal, and specifically a human patient. The pharmaceutical combinations of the invention are also useful for reducing advanced glycated end products (AGEs), ROS, lipid peroxidation, tissue and plasma TNFα and IL6 levels, and for delaying or preventing cardiovascular complications associated with atherosclerosis. Also, the pharmaceutical combinations of this invention are useful for protecting pancreatic β-cells, preventing their impairment or failure and subsequent lower insulin secretion.

It will be understood by the skilled worker that these certain embodiments of the invention are useful for treating a diabetic mammal, preferably a human, whereas other combinations of antioxidants and anti-inflammatory compounds may not be. The particular combination of antioxidant and anti-inflammatory agent, and the efficacy, half-life, absorption, solubility, formulation compatibility, stability, or synergistic or complementary effects of the combination are determined empirically with each combination of particular agents.

Other aspects of this invention provide methods for treating diabetes, particularly Type I and Type II diabetes, as well as diseases and disorders associated with diabetes, including but not limited to atherosclerosis, cardiovascular diseases, inflammatory disorders, nephropathy, neuropathy, and retinopathy, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising an antioxidant agent and an anti-inflammatory agent.

In certain embodiments, this invention provides methods for treating metabolic disorders that include pancreatic β-cell dysfunction, dyslipidemia, hyperglycemia, insulin resistance, metabolic syndrome, LADA, type I diabetes, and type II diabetes, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising an antioxidant agent and an anti-inflammatory agent.

In other embodiments, this invention provides methods for reducing advanced glycated end products (AGEs), ROS, lipid peroxidation, tissue and plasma TNFα and IL6 levels, and for delaying or preventing cardiovascular complications associated with atherosclerosis in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising an antioxidant agent and an anti-inflammatory agent.

The invention thus provides methods for treating diabetes, particularly Type I and Type II diabetes, as well as diseases and disorders associated with diabetes, including but not limited to atherosclerosis, cardiovascular diseases, inflammatory disorders, nephropathy, neuropathy, and retinopathy, in a mammal, particularly a diabetic mammal and particularly a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient.

In certain embodiments, this invention provides methods for treating metabolic disorders that include pancreatic β-cell dysfunction, dyslipidemia, hyperglycemia, insulin resistance, metabolic syndrome, LADA, type I diabetes, and type II diabetes, in a mammal, particularly a diabetic mammal and particularly a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient. The invention particularly provides pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In other embodiments, this invention provides methods for reducing advanced glycated end products (AGEs), ROS, lipid peroxidation, tissue and plasma TNFα and IL6 levels, and for delaying or preventing cardiovascular complications associated with atherosclerosis in a mammal, particularly a diabetic mammal and specifically a human patient in need of such treatment by administering a therapeutically effective amount, particularly a synergistically effective amount of a pharmaceutical composition of a pharmaceutical combination comprising a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient. The invention particularly provides pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

Specific embodiments of such therapeutic methods provided by the invention include methods for treating diabetes, particularly Type I and Type II diabetes, as well as diseases and disorders associated with diabetes, including but not limited to atherosclerosis, cardiovascular diseases, inflammatory disorders, nephropathy, neuropathy, insulin resistance and retinopathy, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. Each of these combinations can optionally comprise one or more pharmaceutically acceptable carriers, diluents or excipients

Additional specific embodiments of such therapeutic methods provided by the invention include methods for treating metabolic disorders that include pancreatic β-cell dysfunction, dyslipidemia, hyperglycemia, insulin resistance, metabolic syndrome, LADA, type I diabetes, and type II diabetes, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising an anti-inflammatory compound including but not limited to an NSAID or a pharmaceutically acceptable salt thereof; N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. Each of these combinations can optionally comprise one or more pharmaceutically acceptable carriers, diluents or excipients

Additional specific embodiments of such therapeutic methods provided by the invention include methods for reducing advanced glycated end products (AGEs), ROS, lipid peroxidation, tissue and plasma TNFα and IL6 levels, and for delaying or preventing cardiovascular complications associated with atherosclerosis in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and a non-steroidal anti-inflammatory drug (NSAID) or a pharmaceutically acceptable salt thereof; N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. Each of these combinations can optionally comprise one or more pharmaceutically acceptable carriers, diluents or excipients

Individual disorders can also be treated using methods provided by the invention, such as diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, and/or insulin resistance. As will be understood by the skilled worker, particular combinations of an antioxidant compound and an anti-inflammatory compound are administered to a mammal, particularly a diabetic mammal, and specifically a human patient in need thereof, for the treatment of such individual diseases or disorders. As provided herein, the methods of the invention comprise the step of administering to a mammal, particularly a diabetic mammal and specifically a human patient, a pharmaceutical compositions for treating diabetes comprising a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient. Particular examples of such combinations are NAC and salicylic acid or a pharmaceutically acceptable salt thereof such as sodium salicylate. Alternative embodiments include but are not limited to combinations of NAC and paracetamol, NAC and ibuprofen, NAC and salsalate, and NAC and diflunisal. Additional particular embodiments include pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

Thus, in certain embodiments, the invention provides methods for treating pancreatic β-cell dysfunction in a patient that includes the step of administering to the patient in need of such treatment a therapeutically effective amount, particularly a synergistically-effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to NSAIDs or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In other embodiments, the invention provides methods for treating dyslipidemia in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In other embodiments, the invention provides methods for treating hyperglycemia in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating insulin resistance in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating metabolic syndrome in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating Type I diabetes in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating Type II diabetes in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating Latent Autoimmune Diabetes of Adulthood (LADA) in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating atherosclerosis in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating cardiovascular diseases in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating inflammatory disorders in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating chronic obstructive pulmonary disease in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating nephropathy in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers

In other embodiments, the invention provides methods for treating neuropathy in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In other embodiments, the invention provides methods for treating retinopathy in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In other embodiments, the invention provides methods for treating metabolic disorders in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In other embodiments, the invention provides methods for treating insulin resistance in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, and an anti-inflammatory compound including but not limited to an NSAID or a pharmaceutically acceptable salt thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In other embodiments, the invention provides methods for reducing advanced glycated end products (AGEs), ROS, lipid peroxidation, tissue and plasma TNFα and IL6 levels, and for delaying or preventing cardiovascular complications associated with atherosclerosis in a patient that includes the step of administering to the patient in need of such treatment a therapeutically effective amount, particularly a synergistically effective amount of a pharmaceutical composition of a pharmaceutical combination comprising thereof, wherein specific examples of such combinations are NAC, alpha-lipoic acid or a pharmaceutically acceptable salt thereof or taurine and salicylic acid or a pharmaceutically acceptable salt thereof such as sodium salicylate. Alternative embodiments include but are not limited to combinations of NAC, alpha-lipoic acid or a pharmaceutically acceptable salt thereof or taurine and paracetamol; NAC, alpha-lipoic acid or a pharmaceutically acceptable salt thereof or taurine and ibuprofen; NAC, alpha-lipoic acid or a pharmaceutically acceptable salt thereof or taurine and salsalate; NAC, alpha-lipoic acid or a pharmaceutically acceptable salt thereof or taurine and dexibuprofen; NAC, alpha-lipoic acid or a pharmaceutically acceptable salt thereof or taurine and dexketoprofen; NAC, alpha-lipoic acid or a pharmaceutically acceptable salt thereof or taurine and HTB, NAC, alpha-lipoic acid or a pharmaceutically acceptable salt thereof or taurine and naproxen; NAC, alpha-lipoic acid or a pharmaceutically acceptable salt thereof or taurine and diclofenac; NAC, alpha-lipoic acid or a pharmaceutically acceptable salt thereof or taurine and sulindac; and NAC, alpha-lipoic acid or a pharmaceutically acceptable salt thereof or taurine and diflunisal. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating pancreatic β-cell dysfunction in a patient that includes the step of administering to the patient in need of such treatment a therapeutically effective amount, particularly a synergistically effective amount of a pharmaceutical composition of a pharmaceutical combinations comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating dyslipidemia in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating hyperglycemia in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating insulin resistance in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating metabolic syndrome in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating Type I diabetes in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating Type II diabetes in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating Latent Autoimmune Diabetes of Adulthood (LADA) in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating atherosclerosis in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating cardiovascular diseases in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating inflammatory disorders in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating chronic obstructive pulmonary disease in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating nephropathy in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating neuropathy in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating retinopathy in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating metabolic disorders in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for reducing advanced glycated end products (AGEs), ROS, lipid peroxidation, tissue and plasma TNFα and IL6 levels, and for delaying or preventing cardiovascular complications associated with atherosclerosis in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for reducing free fatty acids in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for reducing triglycerides in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In particular embodiments, the invention provides methods for treating hyperglycemia in a patient that includes the step of administering to the patient in need of such treatment a therapeutically effective amount, particularly a synergistically effective amount of a pharmaceutical composition of a pharmaceutical combination comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof. In additional particular embodiments, pharmaceutical compositions comprising (R) alpha-lipoic acid or a pharmaceutically acceptable salt thereof, diflunisal or a pharmaceutically acceptable salt thereof, and optionally one or more pharmaceutically acceptable carriers; (R) alpha-lipoic acid or a pharmaceutically acceptable salt thereof, diclofenac or a pharmaceutically acceptable salt thereof, and optionally one or more pharmaceutically acceptable carriers; (R) alpha-lipoic acid or a pharmaceutically acceptable salt thereof, dexibuprofen or a pharmaceutically acceptable salt thereof, and optionally one or more pharmaceutically acceptable carriers; (R) alpha-lipoic acid or a pharmaceutically acceptable salt thereof, dexketoprofen or a pharmaceutically acceptable salt thereof, and optionally one or more pharmaceutically acceptable carriers; (R) alpha-lipoic acid or a pharmaceutically acceptable salt thereof, naproxen or a pharmaceutically acceptable salt thereof, and optionally one or more pharmaceutically acceptable carriers; or (R) alpha-lipoic acid or a pharmaceutically acceptable salt thereof, salicylate or a pharmaceutically acceptable salt thereof, and optionally one or more pharmaceutically acceptable carriers are administered. The invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

This invention also provides pharmaceutically acceptable compositions comprising an antioxidant agent, an anti-inflammatory agent, and at least one pharmaceutically acceptable carrier useful for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, and/or insulin resistance in a mammal, particularly a diabetic mammal, and specifically a human patient. The pharmaceutically acceptable compositions comprising an antioxidant agent, an anti-inflammatory agent, and at least one pharmaceutically acceptable carrier are also useful for reducing AGEs, ROS, lipid peroxidation, tissue and plasma TNFα and IL6 levels, and for delaying or preventing cardiovascular complications associated with atherosclerosis. Also, the pharmaceutically acceptable compositions comprising an antioxidant, an anti-inflammatory agent, and at least one pharmaceutically acceptable carrier are useful for protecting pancreatic β-cells, preventing their impairment or failure and subsequent lower insulin secretion. As provided herein, the pharmaceutically-acceptable compositions comprise a combination of an antioxidant selected from resveratrol, silibinin, alpha-lipoic acid or a pharmaceutically acceptable salt thereof, pterostilbene, N-acetyl cysteine, taurine, probucol, curcumin, alpha-tocopherol and idebenone in combination with an anti-inflammatory selected from sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen in amounts that are therapeutically-effective for treating the disorders disclosed herein in a mammal, particularly a diabetic mammal and specifically a human patient.

In certain embodiments, this invention provides pharmaceutically acceptable compositions comprising N-acetylcysteine, alpha-lipoic acid or a pharmaceutically acceptable salt thereof or taurine, or a pharmaceutically acceptable salt thereof, an anti-inflammatory compound including but not limited to an NSAID or a pharmaceutically acceptable salt thereof, and at least one pharmaceutically acceptable carrier useful for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, and/or insulin resistance. The pharmaceutically acceptable compositions comprising N-acetylcysteine, alpha-lipoic acid or a pharmaceutically acceptable salt thereof or taurine, or a pharmaceutically acceptable salt thereof, an anti-inflammatory compound including but not limited to an NSAID or a pharmaceutically acceptable salt thereof, and at least one pharmaceutically acceptable carrier are also useful for reducing advanced glycated end products (AGEs), ROS, lipid peroxidation, tissue and plasma TNFα and IL6 levels, and for delaying or preventing cardiovascular complications associated with atherosclerosis. Also, the pharmaceutically acceptable compositions comprising N-acetylcysteine, alpha-lipoic acid or a pharmaceutically acceptable salt thereof or taurine, or a pharmaceutically acceptable salt thereof, an anti-inflammatory compound including but not limited to an NSAID or a pharmaceutically acceptable salt thereof, and at least one pharmaceutically acceptable carrier are useful for protecting pancreatic β-cells, preventing their impairment or failure and subsequent lower insulin secretion.

The invention particularly provides such pharmaceutically acceptable compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers. In certain particular embodiments, this invention provides pharmaceutically acceptable compositions comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier useful for treating diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, and/or insulin resistance. The pharmaceutically acceptable compositions comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier are useful for reducing advanced glycated end products (AGEs), ROS, lipid peroxidation, tissue and plasma TNFα and IL6 levels, and for delaying or preventing cardiovascular complications associated with atherosclerosis. Also, the pharmaceutically acceptable compositions comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier are useful for protecting pancreatic β-cells, preventing their impairment or failure and subsequent lower insulin secretion.

In another aspect, this invention provides methods for treating a plurality of diseases and disorders related to dysregulation of glucose homeostatis in a mammal, particularly a diabetic mammal, and specifically a human patient, and specifically diabetes, particularly Type I and Type II diabetes, and diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, and/or insulin resistance. In this aspect, the methods of this invention include the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising an antioxidant agent, an anti-inflammatory agent, and at least one pharmaceutically acceptable carrier.

The invention thus provides methods for treating atherosclerosis, cardiovascular diseases, inflammatory disorders, nephropathy, neuropathy and retinopathy in a mammal, particularly a diabetic mammal, and specifically a human patient, that include the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising an antioxidant agent, an anti-inflammatory agent, and at least one pharmaceutically acceptable carrier.

This invention also provides methods for treating metabolic disorders that include pancreatic β-cell dysfunction, dyslipidemia, hyperglycemia, insulin resistance, metabolic syndrome, LADA, type I diabetes, and type II diabetes, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising an antioxidant agent, an anti-inflammatory agent, and at least one pharmaceutically acceptable carrier.

The invention further provides methods for reducing advanced glycated end products (AGEs), ROS, lipid peroxidation, tissue and plasma TNFα and IL6 levels, and for delaying or preventing cardiovascular complications associated with atherosclerosis in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising an antioxidant agent, an anti-inflammatory agent, and at least one pharmaceutically acceptable carrier.

In certain embodiments of this aspect of the invention are provided methods for treating atherosclerosis, cardiovascular diseases, inflammatory disorders, nephropathy, neuropathy, and retinopathy, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof, an anti-inflammatory compound including but not limited to an NSAID or a pharmaceutically acceptable salt thereof, and at least one pharmaceutically acceptable carrier.

In certain embodiments of this aspect of the invention are provided methods for treating metabolic disorders that include pancreatic β-cell dysfunction, dyslipidemia, hyperglycemia, insulin resistance, metabolic syndrome, LADA, type I diabetes, and type II diabetes, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising an N-acetylcysteine, alpha-lipoic acid or a pharmaceutically acceptable salt thereof or taurine, or a pharmaceutically acceptable salt thereof, an anti-inflammatory compound including but not limited to an NSAID or a pharmaceutically acceptable salt thereof, and at least one pharmaceutically acceptable carrier.

In certain embodiments of this aspect of the invention are provided methods for reducing advanced glycated end products (AGEs), ROS, lipid peroxidation, tissue and plasma TNFα and IL6 levels, and for delaying or preventing cardiovascular complications associated with atherosclerosis in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof, an anti-inflammatory compound including but not limited to an NSAID or a pharmaceutically acceptable salt thereof, and at least one pharmaceutically acceptable carrier.

In particular embodiments, this invention provides methods for treating atherosclerosis, cardiovascular diseases, inflammatory disorders, nephropathy, neuropathy, and retinopathy, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating metabolic disorders that include pancreatic β-cell dysfunction, dyslipidemia, hyperglycemia, insulin resistance, metabolic syndrome, LADA, type I diabetes, and type II diabetes, in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising an N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, or a pharmaceutically acceptable salt thereof and diflunisal, wherein each such combination further comprises at least one pharmaceutically acceptable carrier.

In particular embodiments, this invention provides methods for reducing advanced glycated end products (AGEs), ROS, lipid peroxidation, tissue and plasma TNFα and IL6 levels, and for delaying or preventing cardiovascular complications associated with atherosclerosis in a mammal, particularly a diabetic mammal, and specifically a human patient that includes the step of administering to the mammal, particularly a diabetic mammal, and specifically a human patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

This invention also provides methods for treating pancreatic β-cell dysfunction in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating dyslipidemia in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating hyperglycemia in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating insulin resistance in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating metabolic syndrome in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating Type I diabetes in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating Type II diabetes in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating Latent Autoimmune Diabetes of Adulthood (LADA) in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating atherosclerosis in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating cardiovascular diseases in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating inflammatory disorders in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating chronic obstructive pulmonary disease in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating nephropathy in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating neuropathy in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating retinopathy in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for treating metabolic disorders in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

This invention also provides methods for reducing advanced glycated end products (AGEs), ROS, lipid peroxidation, tissue and plasma TNFα and IL6 levels, and for delaying or preventing cardiovascular complications associated with atherosclerosis in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, an anti-inflammatory compound including but not limited to an NSAID or a pharmaceutically acceptable salt thereof, and at least one pharmaceutically acceptable carrier.

In particular embodiments, this invention provides methods for treating pancreatic β-cell dysfunction in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating dyslipidemia in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating hyperglycemia in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating insulin resistance in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating metabolic syndrome in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating Type I diabetes in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating Type II diabetes in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating Latent Autoimmune Diabetes of Adulthood (LADA) in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating atherosclerosis in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating cardiovascular diseases in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating inflammatory disorders in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating chronic obstructive pulmonary disease in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating nephropathy in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating neuropathy in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating retinopathy in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating metabolic disorders in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for reducing advanced glycated end products (AGEs), ROS, lipid peroxidation, tissue and plasma TNFα and IL6 levels, and for delaying or preventing cardiovascular complications associated with atherosclerosis in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for reducing free fatty acids in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for reducing triglycerides in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In particular embodiments, this invention provides methods for treating hyperglycemia in a patient that includes the step of administering to the patient in need of such treatment a therapeutically effective amount, particularly a synergistically effective amount of a pharmaceutical composition of a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier.

In each of the foregoing methods, the invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In another aspect, this invention provides a use for a pharmaceutical combination comprising N-acetylcysteine, alpha-lipoic acid or a pharmaceutically acceptable salt thereof or taurine, and an anti-inflammatory compound including but not limited to an NSAID for preparing, or for the manufacture of, a medicament for treating diabetes, particularly Type I and Type II diabetes, and diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease, in a mammal, particularly a diabetic mammal, and specifically a human patient. This invention also provides a use for pharmaceutical combinations comprising N-acetylcysteine, alpha-lipoic acid or a pharmaceutically acceptable salt thereof or taurine, and an anti-inflammatory compound including but not limited to an NSAID, for preparing, or for the manufacture of, a medicament for reducing AGEs, ROS, lipid peroxidation, tissue and/or plasma TNFα and IL6 levels, and for delaying or preventing cardiovascular complications associated with atherosclerosis in a mammal, particularly a diabetic mammal, and specifically a human patient.

In particular embodiments, this invention provides a use for a pharmaceutical combination comprising N-acetylcysteine, alpha-lipoic acid or a pharmaceutically acceptable salt thereof or taurine, and sodium salicylate, for preparing, or for the manufacture of, a medicament for treating diabetes, particularly Type I and Type II diabetes, and diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease, in a mammal, particularly a diabetic mammal, and specifically a human patient. This invention also provides a use for a pharmaceutical combinations comprising N-acetylcysteine, alpha-lipoic acid or a pharmaceutically acceptable salt thereof or taurine, and sodium salicylate, for preparing, or for the manufacture of, a medicament for reducing AGEs, ROS, lipid peroxidation, tissue and/or plasma TNFα and IL6 levels, and for delaying or preventing cardiovascular complications associated with atherosclerosis in a mammal, particularly a diabetic mammal, and specifically a human patient.

In further particular embodiments, this invention provides a use for a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier, for preparing, or for the manufacture of, a medicament for treating diabetes, particularly Type I and Type II diabetes, and diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease, in a mammal, particularly a diabetic mammal, and specifically a human patient. This invention also provides a use for a pharmaceutically acceptable composition comprising N-acetylcysteine, alpha-lipoic acid or a pharmaceutically acceptable salt thereof or taurine, or a pharmaceutically acceptable salt thereof and salicylic acid or a pharmaceutically acceptable salt thereof such as sodium salicylate; N-acetylcysteine, alpha-lipoic acid or a pharmaceutically acceptable salt thereof or taurine, or a pharmaceutically acceptable salt thereof and ibuprofen; N-acetylcysteine, alpha-lipoic acid or a pharmaceutically acceptable salt thereof or taurine, or a pharmaceutically acceptable salt thereof and paracetomol or a pharmaceutically acceptable salt thereof; N-acetylcysteine, alpha-lipoic acid or a pharmaceutically acceptable salt thereof or taurine, or a pharmaceutically acceptable salt thereof and salsalate or a pharmaceutically acceptable salt thereof; N-acetylcysteine, alpha-lipoic acid or a pharmaceutically acceptable salt thereof or taurine, or a pharmaceutically acceptable salt thereof and sulindac or a pharmaceutically acceptable salt thereof; N-acetylcysteine, alpha-lipoic acid or a pharmaceutically acceptable salt thereof or taurine, or a pharmaceutically acceptable salt thereof and dexketoprofen or a pharmaceutically acceptable salt thereof; N-acetylcysteine, alpha-lipoic acid or a pharmaceutically acceptable salt thereof or taurine, or a pharmaceutically acceptable salt thereof and dexibupofen or a pharmaceutically acceptable salt thereof; N-acetylcysteine, alpha-lipoic acid or a pharmaceutically acceptable salt thereof or taurine, or a pharmaceutically acceptable salt thereof and HTB or a pharmaceutically acceptable salt thereof; N-acetylcysteine, alpha-lipoic acid or a pharmaceutically acceptable salt thereof or taurine, or a pharmaceutically acceptable salt thereof and naproxen or a pharmaceutically acceptable salt thereof; N-acetylcysteine, alpha-lipoic acid or a pharmaceutically acceptable salt thereof or taurine, or a pharmaceutically acceptable salt thereof and diclofenac or a pharmaceutically acceptable salt thereof; N-acetylcysteine, alpha-lipoic acid or a pharmaceutically acceptable salt thereof or taurine, or a pharmaceutically acceptable salt thereof and diflunisal or a pharmaceutically acceptable salt thereof, wherein each such combination further comprises at least one pharmaceutically acceptable carrier, for preparing, or for the manufacture of, a medicament for reducing AGEs, ROS, lipid peroxidation, tissue and/or plasma TNFα and IL6 levels, and for delaying or preventing cardiovascular complications associated with atherosclerosis in a mammal, particularly a diabetic mammal, and specifically a human patient.

In each of the foregoing methods, the invention particularly provides such methods using pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers.

In another aspect, this invention provides methods for treating any of the aforementioned diseases and disorders: adipocyte dysfunction related diseases, carbohydrate metabolism related diseases, vascular diseases, neurodegenerative diseases, cancers, arthritis, osteoarthritis, spondylitis, bone resorption diseases, sepsis, septic shock, chronic pulmonary inflammatory disease, fever, periodontal diseases, ulcerative colitis, pyresis, Alzheimer's disease, Parkinson's diseases, cystic fibrosis, dysfunctions of the immune system, stroke, multiple sclerosis, migraine, pain, inflammatory eye conditions including uveitis, glaucoma and conjunctivitis, degenerative bone or joint conditions including osteoarthritis, rheumatoid arthritis, rheumatoid spondylitis, gouty arthritis ankylosing spondylitis, psoriatic arthritis and other arthritic conditions, as well as inflamed joints, chronic inflammatory skin conditions, including allergic lesions, lichen planus, pityriasis rosea, eczema, psoriasis, and dermatitis, diseases and disorders of the gastrointestinal tract, including inflammatory bowel disease, Crohn's disease, atrophic gastritis, gastritis varialoforme, ulcerative colitis, coeliac disease, regional ileitis, peptic ulceration, particularly irritable bowel syndrome, reflux oesophagitis, and damage to the gastrointestinal tract resulting from infections, for example, by Helicobacter pylori, inflammatory lung disorders such as asthma, bronchitis, particularly chronic obstructive pulmonary disease, farmer's lung, acute respiratory distress syndrome; bacteraemia, endotoxaemia (septic shock), aphthous ulcers, gingivitis, pyresis, particularly pain, including inflammatory pain, neuropathic pain, acute pain or pain of a central origin; meningitis and pancreatitis, and other conditions associated with inflammation, central nervous system inflammatory conditions and diseases, including ischaemia-reperfusion injury associated with ischemic stroke; vascular diseases, such as atheromatous and nonatheromatous, ischemic heart disease, and Raynaud's Disease and Phenomenon in a mammal, particularly a diabetic mammal, and specifically a human patient comprising administering to the mammal, particularly a diabetic mammal, and specifically a human patient in need of such treatment a therapeutically effective amount, particularly a synergistically effective amount of a pharmaceutical composition of a pharmaceutical combination comprising an antioxidant agent, an anti-inflammatory agent, and optionally at least one other anti-diabetic agent. In certain embodiments, this invention provides uses for pharmaceutical combination for preparing, or for the manufacture of, a medicament for treating the diseases/disorders listed above.

In another aspect, this invention provides methods for treating any of the aforementioned diseases and disorders adipocyte dysfunction related diseases, carbohydrate metabolism related diseases, vascular diseases, neurodegenerative diseases, cancers, arthritis, osteoarthritis, spondylitis, bone resorption diseases, sepsis, septic shock, chronic pulmonary inflammatory disease, fever, periodontal diseases, ulcerative colitis, pyresis, Alzheimer's disease, Parkinson's diseases, cystic fibrosis, dysfunctions of the immune system, stroke, multiple sclerosis, migraine, pain, inflammatory eye conditions including uveitis, glaucoma and conjunctivitis, degenerative bone or joint conditions including osteoarthritis, rheumatoid arthritis, rheumatoid spondylitis, gouty arthritis ankylosing spondylitis, psoriatic arthritis and other arthritic conditions, as well as inflamed joints, chronic inflammatory skin conditions, including allergic lesions, lichen planus, pityriasis rosea, eczema, psoriasis, and dermatitis, diseases and disorders of the gastrointestinal tract, including inflammatory bowel disease, Crohn's disease, atrophic gastritis, gastritis varialoforme, ulcerative colitis, coeliac disease, regional ileitis, peptic ulceration, particularly irritable bowel syndrome, reflux oesophagitis, and damage to the gastrointestinal tract resulting from infections, for example, by Helicobacter pylori, inflammatory lung disorders such as asthma, bronchitis, particularly chronic obstructive pulmonary disease, farmer's lung, acute respiratory distress syndrome; bacteraemia, endotoxaemia (septic shock), aphthous ulcers, gingivitis, pyresis, particularly pain, including inflammatory pain, neuropathic pain, acute pain or pain of a central origin; meningitis and pancreatitis, and other conditions associated with inflammation, central nervous system inflammatory conditions and diseases, including ischaemia-reperfusion injury associated with ischemic stroke; vascular diseases, such as atheromatous and nonatheromatous, ischemic heart disease, and Raynaud's Disease and Phenomenon in a mammal, particularly a diabetic mammal, and specifically a human patient comprising administering to the mammal, particularly a diabetic mammal, and specifically a human patient in need of such treatment a therapeutically effective amount, particularly a synergistically effective amount of a pharmaceutical composition of a pharmaceutically acceptable composition comprising an antioxidant agent, an anti-inflammatory agent, optionally at least one other anti-diabetic agent, and at least one pharmaceutically acceptable carrier. In certain embodiments, this invention provides uses for pharmaceutical combination for preparing, or for the manufacture of, a medicament for treating the diseases/disorders listed above.

The antioxidant agents and anti-inflammatory of this invention may be administered to a mammal, particularly a diabetic mammal, and specifically a human patient combined as a pharmaceutical combination or as a pharmaceutical composition. This invention also includes pharmaceutical combinations wherein the antioxidant and anti-inflammatory agents are administered at the same time, or nearly the same time, as separate agents. Combinations of antioxidants and anti-inflammatory agents according to this invention are provided in ratios of from about 30:1 to about 1:30, alternatively about 20:1 to about 1:20 and in further alternatives from about 10:1 to about 1:10.

The term “anti-diabetic agent” as used herein means any one of metformin, glyburide, glimepiride, glipyride, glipizide, chlorpropamide, gliclazide, acarbose, miglitol, pioglitazone, troglitazone, rosiglitazone, insulin, isaglitazone, repaglinide, and nateglinide. In accordance with this invention, the pharmaceutical combinations or pharmaceutically acceptable compositions of this invention optionally include at least one anti-diabetic agent. Preferably, one anti-diabetic agent is optionally combined with the pharmaceutical combinations and pharmaceutically acceptable compositions of this invention.

The term “anti-inflammatory agent” as used herein means any one of sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen.

The term “antioxidant agent” as used herein means any one of resveratrol, silibinin, alpha-lipoic acid or a pharmaceutically acceptable salt thereof, pterostilbene, N-acetyl cysteine, taurine, probucol, curcumin, alpha-tocopherol and idebenone.

The term “N-acetylcysteine, or NAC” as used herein includes esters and amides of N-acetylcysteine. Representative esters and amides of N-acetylcysteine, include, but are not limited to, methyl N-acetylcysteinate, ethyl N-acetylcysteinate, isopropyl N-acetylcysteinate, propyl N-acetylcysteinate, tert-butyl N-acetylcysteinate, and N²-acetylcysteinamide. Further, the term “N-acetylcysteine” encompasses the (L) form, the (D) form, and mixtures or racemates thereof, wherein the (L) form is the preferred form of N-acetylcysteine.

The term “NSAID” as used herein means non-steroidal anti-inflammatory drug. NSAID agents are a subset of anti-inflammatory agents and include any one of the following sulindac, salicylic acid, diflunisal, 2-hydroxy-4-trifluoromethylbenzoic acid (HTB), salsalate, naproxen, paracetamol, diclofenac, ibuprofen, dexibuprofen and dexketoprofen.

Combinations according to the invention include at least any anti-oxidant that is N-acetylcysteine, resveratrol, silibinin, α-lipoic acid, particularly (R)-α-lipoic acid, idebenone, taurine, probucol, curcumin, pterostilbene or α-tocopherol, with at least any anti-inflammatory that is sulindac, salicylic acid or salts thereof, diflunisal, HTB, salsalate, naproxen, paracetomol, dexibuprofen, dexketoprofen, ibuprofen, or diclofenac. Particularly-advantageous embodiments of the combinations of this invention are combinations of the antioxidants N-acetylcysteine, alpha-lipoic acid (particularly (R)-alpha-lipoic acid) or taurine with anti-inflammatories sulindac, salicylic acid, diflunisal, 2-hydroxy-4-trifluoromethylbenzoic acid (HTB), naproxen, paracetamol, diclofenac, dexibuprofen or dexketoprofen. Particular embodiments of the combinations of the invention include the following:

TABLE I
Antioxidants and anti-inflammatories screened at four-five different
concentrations in the INS-1E β-cell assay (set forth below) showing
concentrations that reduced apoptosis promoted by high (11 mM) glucose
and high (0.4 mM) palmitate concentrations

| | | |
|---|---|---|
| Compound | Concentration range | Combination concentrations |

Antioxidants

| N-acetylcysteine | 0.1-3 mM | 1 mM, 1.5 | mM |
|---|---|---|---|
| Taurine | 0.1-3 mM | 3 | mM |
| α-lipoic acid | 0.01 mM-1 mM | 100, 250, 500 | μM |
| Curcumine | 0.001-0.5 mM | N.D. | |
| Silibinin | 0.001-0.5 mM | N.D. | |
| Idebenone | 0.01 mM-1 mM | 50 | μM |
| Pterostilbene | 0.01 mM-1 mM | 10 | μM |
| α-tocopherol | 0.01 mM-1 mM | 1 | mM |
| Resveratrol | 0.01 mM-1 mM | 10, 50 | μM |

Anti-inflammatories

| | | | |
|---|---|---|---|
| Salicylate | 0.01 mM-1 mM | 100, 250, 500 | μM |
| Paracetomol | 0.01 mM-1 mM | 250 | μM |
| HTB | 0.01 mM-1 mM | 50 | μM |
| Dexibuprofen | 0.01 mM-1 mM | 100, 250, 500 | μM |
| Diflunisol | 0.01 mM-1 mM | 50, 100 | μM |
| Naproxen | 0.01 mM-1 mM | 100, 250, 500 | μM |
| Dexketoprofen | 0.01 mM-1 mM | 100, 250, 500 | μM |
| Diclofenac | 0.01 mM-1 mM | 100, 150 | μM |
| Sulindac | 0.01 mM-1 mM | 250 | μM |

Particular combinations providing at least a 30% inhibition of apoptosis in the INS-1E β-cell assay set forth below included:

α-lipoic acid (0.1 mM) and salicylate (0.5-1 mM)

α-lipoic acid (0.1 mM) and dexibuprofen (0.5-1 mM)

α-lipoic acid (0.1 mM) and dexketoprofen (0.5-1 mM)

α-lipoic acid (0.1 mM) and diclofenac (0.1 mM)

Particular combinations providing protection against insulin resistance in mouse 3T3-L1 adipocytes as described below include:

N-acetylcysteine (1.5 mM) and diflunisal (25 μM)

N-acetylcysteine (1.5 mM) and diclofenac (25 μM)

N-acetylcysteine (1.5 mM) and dexketoprofen (25 μM)

N-acetylcysteine (1.5 mM) and dexibuprofen (100 μM)

N-acetylcysteine (1.5 mM) and salicylate (50 μM)

### Pharmaceutical Compositions

This invention also provides pharmaceutical compositions that comprise compounds of this invention formulated together with one or more non-toxic pharmaceutically acceptable carriers. The pharmaceutical compositions may be specially formulated for oral administration in solid or liquid form, for parenteral injection, or for rectal administration. The invention particularly provides pharmaceutical compositions that comprise lipoic acid, preferrably (R) lipoic acid, in combination with one or more anti-inflammatories selected from the group consisting of diflunisal, diclofenac, dexibuprofen, dexketoprofen, naproxen, and salicylate, optionally formulated together with one or more non-toxic pharmaceutically acceptable carriers. The pharmaceutical compositions may be specially formulated for oral administration in solid or liquid form, for parenteral injection, or for rectal administration.

The term “pharmaceutically acceptable carrier” as used herein means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. This invention provides pharmaceutical compositions which comprise compounds of the invention formulated together with one or more non-toxic pharmaceutically acceptable carriers. The pharmaceutical compositions can be formulated for oral administration in solid or liquid form, for parenteral injection or for rectal administration.

The pharmaceutical compositions of this invention can be administered to humans (patients) and other mammals orally, rectally, parenterally, intracisternally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of this invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more pharmaceutically acceptable carriers as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert pharmaceutically acceptable carrier such as sodium citrate or calcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of this invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of this invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

The phrase “therapeutically effective amount” of the compound of this invention means a sufficient amount of the compound to treat metabolic disorders, at a reasonable benefit/risk ratio applicable to any medical treatment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Actual dosage levels of active ingredients in the pharmaceutical compositions of this invention can be varied so as to obtain an amount of the active compound(s) which is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated.

The total daily dose of the compounds of this invention administered to a mammal, and particularly a diabetic mammal and specifically a human patient, from about 0.03 to about 50 mg/kg/day. For purposes of oral administration, more preferable doses can be in the range of from about 0.1 to about 50 mg/kg/day. If desired, the effective daily dose can be divided into multiple doses for purposes of administration, e.g. two to four separate doses per day. Specifically, antioxidants of the invention can be administered at dosages from about 100 mg to 3000 mg per day, and for anti-inflammatory compounds at dosages from about 250 mg to 3500 mg per day.

The term “pharmaceutically acceptable salt,” as used herein, means a positively-charged inorganic or organic cation that is generally considered suitable for human consumption. Examples of pharmaceutically acceptable cations are alkali metals (lithium, sodium and potassium), magnesium, calcium, ferrous, ferric, ammonium, alkylammonium, dialkylammonium, trialkylammonium, tetraalkylammonium, diethanolammonium, and choline. Cations may be interchanged by methods known in the art, such as ion exchange. Where compounds of this invention are prepared in the carboxylic acid form, addition of a base (such as a hydroxide or a free amine) will yield the appropriate salt form.

This invention contemplates pharmaceutically active metabolites formed by in vivo biotransformation of, for example, methyl N-acetylcysteinate, ethyl N-acetylcysteinate, isopropyl N-acetylcysteinate, propyl N-acetylcysteinate, tert-butyl N-acetylcysteinate, and N²-acetylcysteinamide, to N-acetyl cysteine. A thorough discussion of biotransformation is provided in (Goodman and Gilman's, The Pharmacological Basis of Therapeutics, seventh edition).

All patents, patent applications, and literature references cited in the specification are herein incorporated by reference in their entirety for any purpose.

### EXAMPLES

### Experimental Methods

### Anti-Oxidants/Anti-Inflammatories

N-Acetyl-cysteine, Sodium Salicylate, Taurine, alpha-Tocopherol, Sodium Diclofenac, Dexketoprofen, Naproxen, Curcumine, Silibinin, Idebenone, Pterostilbene, Sulindac, Paracetamol and DMSO were purchased from Sigma (Sigma Aldrich, St. Louis, Mo., USA), Diflunisal and dexibuprofen, were purchase from Galchimia, S. L. (Galchimia S. L., A Coruñ a, Spain), Resveratrol was purchased from Sequoia Research Products Limited (Sequoia Research Products Ltd, Pangbourne, United Kingdom). (R)-α-lipoic acid was purchased from TCI (TCI Europe, Zwijndrecht, Belgium). 2-hydroxy-4-trifluoromethylbenzoic acid (HTB) was purchased from Matrix Scientific (Matrix Scientific, Columbia, S.C., USA). PBS was purchased from Invitrogen (Invitrogen S.A., Barcelona, Spain). N-Acetyl-cysteine, Sodium Salicylate, Taurine, Paracetamol and Naproxen were dissolved in PBS and the pH adjusted with NaOH 6N until pH 7. Curcumine, Idebenone, Diflunisal, Sulindac and Pterostilbene were dissolved in DMSO. Dexibuprofen, Dexketoprofen, Diflunisal and HTB were dissolved in a mix PBS/DMSO 1:1. (R)-α-lipoic acid was dissolved in NaCl 0.9% and the pH adjusted with NaOH 6N and HCl 30% until pH 7.4.

### In Vitro INS-1E β-Cell Assay

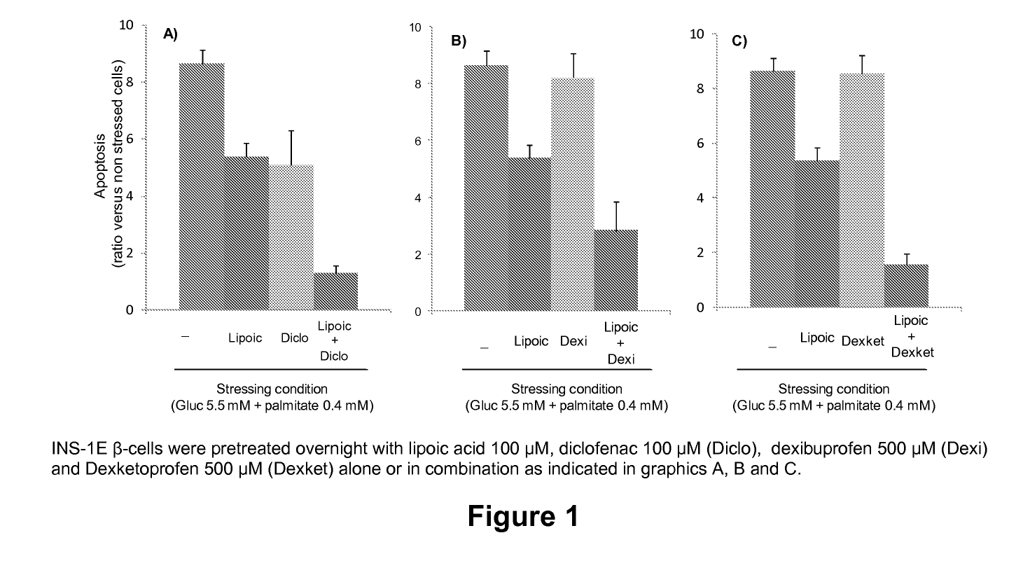

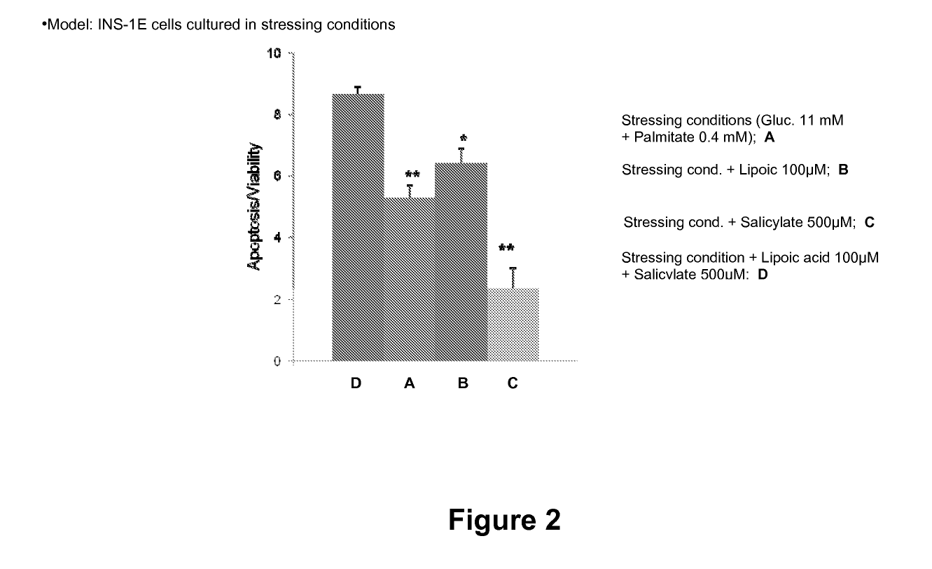

INS-1E β-cells were cultivated in the presence of a high glucose concentration (11 mM) and a high palmitate concentration (0.4 mM bound to BSA 0.5%) in order to promote glucotoxicity and lipotoxicity. The combination of both stressors promoted β-cell apoptosis. The capacity of protecting β-cells with different combinations of antioxydants and anti-inflammatory agents were tested using these stressing conditions that reflect the pathophysiological conditions implicated in pancreatic dysfunction related to diabetes onset. INS-1E cells were seeded at a density of 80,000 cells/cm² in 96 wells plates 4 days before the beginning of the treatment. At 60-80% of confluence, cells were fasted with RPMI 5 mM of glucose+FBS 10%. 8 h later, antioxidants and anti-inflammatory agents, specifically (R) lipoic acid, naproxen, dexketoprofen, diclofenac, or diflunisal, alone or in combination were added overnight at the indicated concentrations; specific concentrations are set forth in FIGS. 1 and 2. The day after, fasting medium was changed by the stressing medium (glucose 25 mM+palmitate 0.4 mM bound to BSA 0.5%). Medium, and tested agents when present, were changed every 24 h. 48 h after the addition of the stressing medium, apoptosis was measured with Apo-One Homogeneous Caspase 3/7 Assay (Promega) which determines the activity of caspase 3 and 7. Cells were frozen at −80° C. for 2 hours, defrosted at room temperature and incubated in the presence of 100 μl of caspase reactive for 20 hours. Resulting fluorescence was read at 485/530 (excitation/emission wave length). The background apoptosis, in absence of stressing condition, was determined with INS 1E-cells cultured in the presence of fasting medium (RPMI 5 mM glucose+FBS 10%). Staurosporine 0.2% in the presence of 0.5% BSA was used as a positive control of apoptosis.

### Animals

Male cd-1 mice weighing 25-30 g were purchased from Charles River Laboratories Spain. 5-weeks old male mice C57BL/Ks bearing the db/db mutation (The Jackson Laboratories) and 7-weeks old male mice C57BL/6 bearing the ob/ob mutation were purchased from Charles River Laboratories Spain (Sant Cugat del Vallès, Spain). The animals were housed in animal quarters at 22° C. with a 12-h light/12-h dark cycle and fed ad libitum.

### Chronic Treatment in Db/Db and Ob/Ob Mice

The animals were treated with the indicated drugs for four weeks. The administration route of in vivo administered drugs is indicated for each treatment in the text of the report. The glycemia levels were determined at 9:00 a.m. in blood from the Tail Vein using a rapid glucose analyzer (Accu-Chek Aviva; Roche) 3 times per week and body weight was measured also. Food and water intake were measured twice a week. Glycemia fasting levels were determined at 9:00 a.m. in blood from the Tail Vein after an overnight fasting using a rapid glucose analyzer. At the end of four weeks, the mice were sacrificed, in feeding state, with CO₂ euthanasia and the blood was extracted from the Inferior Cave Vein using heparin as an anticoagulant and maintained at 4° C. until plasma preparation.

### Intraperitoneal Insulin Tolerance Test.

At the third week of treatment, an Insulin Tolerance Test was done to the mice in the feeding state. The animals received an i.p. injection of Insulin 2 UI/kg (Humulin®). After the Insulin injection, glycemia levels were determined using a rapid glucose analyzer at the indicated time in blood from the Tail Vein.

### Intraperitoneal Glucose Tolerance Test.

At the fourth week, a Glucose Tolerance Test was done to the mice after an overnight fasting. The animals received an i.p. injection of Glucose 0.5 g/kg (Glucosmon 50®). After the Glucose injection, glycemia levels were determined using a rapid glucose analyzer in blood at the indicated time from the Tail Vein.

In Vivo β-Cell Protection Model β-cell destruction was induced in cd-1 male mice after 3 hours of fasting by a single intra-peritoneal (i.p.) injection of a freshly prepared solution of alloxan 200 mg/kg (Sigma-Aldrich, San Luis, Mo.) that was dissolved in NaCl 0.9%. One single intra-peritoneal drug administration was given one hour before the Alloxan administration. Animals received the different drugs dissolved in PBS pH 7.4 and the animals that did not receive any drug were injected with vehicle, PBS pH 7.4. At the end of the treatment, day 4, animals were sacrificed and the plasma collected and kept at −20° C. until used.

### Biochemical Parameters

The circulating glucose concentration was determined by a rapid glucose analyzer (Accu-Chek Aviva; Roche). Plasma triglycerides and non esterified fatty acids were determined with standard colorimetric methods (Biosystems, Barcelona, Spain, and Wako Chemicals, Neuss, Germany, respectively). Plasma insulin concentration was determined by enzyme-linked immunosorbent assay method (CrystalChem, Downers Grove, Ill.). Total pancreas insulin content was determined after extraction of insulin from pancreas homogenates with a mixture of Ethanol (70%)/HCl (0.15 N).

### Statistical Analysis.

Statistical comparisons between groups were established by two-way ANOVA or one-way ANOVA using Prism 4 (GraphPad, San Diego, Calif.). A p value of less than 0.05 was considered to be statistically significant. Statistically significant differences are indicated as follow: *, P<0.05; **, P<0.01; ***, P<0.001.

### Biological Data

The animals for Examples 1 and 2 were housed in animal quarters at 22° C. with a 12-h light/12-h dark cycle and were fed ad libitum. All procedures used were approved by the animal ethical committee of the Scientific Park of Barcelona-University of Barcelona.

N-Acetyl-cysteine and Sodium Salicylate were purchased from Sigma-Aldrich (St. Louis, Mo., USA) and PBS was purchased from Invitrogen.

### Example 1

### Alloxan Induced Beta Cell Destruction Model

Male cd-1 mice weighing 25-30 g were purchased from Charles River Laboratories Spain (Sant Cugat del Vallès, Spain). Pancreatic beta cell destruction was induced in the cd-1 mice after 3 hours of fasting by a single intraperitoneal injection of a freshly prepared solution of alloxan 200 mg/kg (Sigma-Aldrich, San Luis, Mo.) that was dissolved in NaCl 0.9%. Single drug intraperitoneal administration was 1 hour before the alloxan administration. Animals received N-acetylcysteine 0.19 mmol/kg alone, Sodium Salicylate 0.75 mmol/kg alone, or the combination of both. The control group was injected with the vehicle, PBS at pH 7.4. Glycemia was measured on arterio-venous blood collected from the tail vessels between 9:00 and 10:00 am on day 0 (day of drug administration) to day 4. The circulating glucose concentration were determined by a rapid glucose analyzer (Accu-Chek Aviva; Roche).

Statistical comparisons between groups were established by two-way ANOVA using Prism 4 (GraphPad, San Diego, Calif.). A p value of less than 0.05 was considered to be statistically significant.

### Example 2

### Chronic Treatment of Db/Db Mice

Eight week old Male mice C57BL/Ks bearing the db/db mutation (The Jackson Laboratories) were purchased from Charles River Laboratories Spain (Sant Cugat del Vallés, Spain). The db/db mice were treated i.p. with N-acetylcysteine alone, sodium salicylate alone, or the combination of N-acetylcysteine and sodium salicylate at 0.75 mmol/kg/day. After 4 weeks of treatment, mice were sacrificed with CO₂ euthanasia and blood was extracted from the inferior cave vein and maintained at 4° C. until plasma obtention by centrifugation (13 000 g) for 15 min at 4° C., and stored at −80° C. until use for the measure of plasma triglycerides and nonesterified fatty acids. Plasma triglycerides and nonesterified fatty acids were determined with standard colorimetric methods (Biosystems, Barcelona, Spain, and Wako Chemicals, Neuss, Germany, respectively).

Statistical comparisons between groups were established by two-way ANOVA using Prism 4 (GraphPad, San Diego, Calif.). A p value of less than 0.05 was considered to be statistically significant.

The results of Experiments 1 and 2 show that the combination of an antioxidant agent and an anti-inflammatory agent is more effective at reducing glucose, free fatty acids, and triglyceride levels than an antioxidant alone or an anti-inflammatory alone. The additive or synergistic effect improves anti-diabetic effect while reducing side effects associated with monotherapy. In particular, treatment with the pharmaceutical combination of N-acetylcysteine and sodium salicylate improves anti-diabetic effects while lowering the risk of gastric bleeding and/or tinnitus associated with salicylic acid.

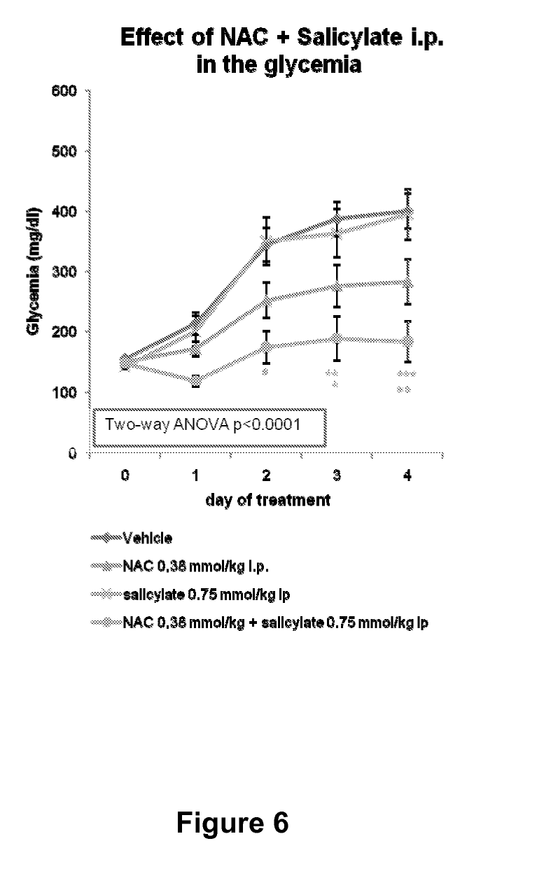

Further exemplary experimental results showing the efficacy of certain embodiments of the combinations of the invention are set forth in the drawings. As set forth in greater detail in Example 1, N-acetyl cysteine alone, sodium salicylate alone, and the combination of N-acetyl cysteine and sodium salicylate was administered over a 5-day period cd-1 mice in which pancreatic beta cell destruction was induced using alloxan. As shown in FIG. 6, glucose levels were reduced (expressed as glycemia in mg/mL) by administration of the combination of N-acetyl cysteine and sodium salicylate compared with treatment with vehicle, N-acetylcysteine or sodium salicylate separately, having a significance of p<0.0001 by two-way ANOVA analysis.

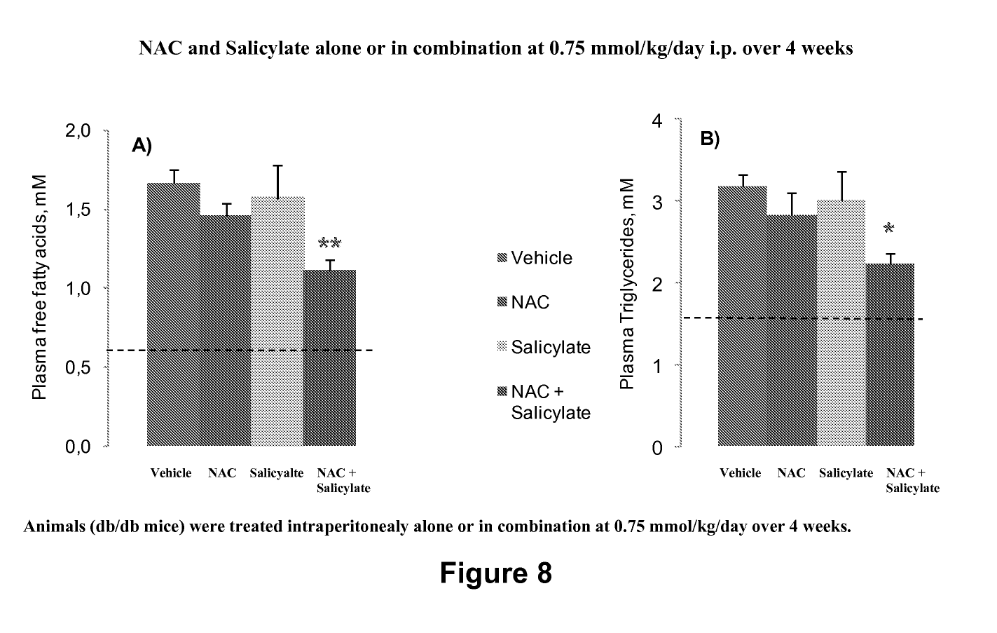

These results of acute administration of N-acetyl cysteine and sodium salicylate were consistent with the results of chronic administration of N-acetyl cysteine and sodium salicylate to db/db mice over a four-week course of treatment on free fatty acids and triglycerides (markers of metabolic syndrome). As shown in FIG. 8, administration of the combination of N-acetyl cysteine and sodium salicylate showed a statistically-significant decrease in free fatty acid concentration in the systemic circulation of db/db mice compared with treatment with vehicle, N-acetylcysteine or sodium salicylate separately. Similarly, administration of the combination of N-acetyl cysteine and sodium salicylate showed a statistically-significant decrease in triglycerides in the systemic circulation of db/db mice compared with treatment with vehicle, N-acetylcysteine or sodium salicylate separately. These experiments are set forth in greater detail in Example 2.

The effects of combinations of (R)-alpha-lipoic acid with diclofenac, dexibuprofen and dexketoprofen on cellular stress on pancreatic beta-cell function was investigated using an in vitro model system, INS-1E beta-cells. Cells were incubated in the presence of glucose (11 mM) and palmitate concentration (0.4 mM bound to BSA 0.5%) at concentrations associated with glucotoxic and lipotoxic stress associated with beta-cell apoptosis. Cells were pretreated overnight with (R) alpha-lipoic acid or a pharmaceutically acceptable salt thereof alone (100 μM), diclofenac (100 μM), dexibuprofen (500 μM), or dexketoprofen (500 μM) alone or in combinations of (R) alpha-lipoic acid or a pharmaceutically acceptable salt thereof (100 μM) and diclofenac (100 μM), (R) alpha-lipoic acid or a pharmaceutically acceptable salt thereof (100 μM) and dexibuprofen (500 μM), or (R) alpha-lipoic acid or a pharmaceutically acceptable salt thereof (100 μM) and dexketoprofen (500 μM). As shown in FIG. 1, incubation of the cells with each of these combinations showed a much greater reduction in apoptosis that administration of (R) alpha-lipoic acid or a pharmaceutically acceptable salt thereof, declofenac, dexibuprofen or dexketoprofen alone.

These experiments were repeated using the same cells under the same apoptosis-inducing (“stressing”) conditions in a comparison of the effects of (R) alpha-lipoic acid or a pharmaceutically acceptable salt thereof alone (100 μM), sodium salicylate alone (500 μM), or the combination of (R) alpha-lipoic acid or a pharmaceutically acceptable salt thereof (100 μM) and sodium salicylate (500 μM). The results of these experiments, shown in FIG. 2, indicated that the combination of (R) alpha-lipoic acid or a pharmaceutically acceptable salt thereof (100 μM) and sodium salicylate (500 μM) had a significantly greater increase in cellular viability under stressing conditions than incubation of INS-1E-beta-cells with (R) alpha-lipoic acid or a pharmaceutically acceptable salt thereof or sodium salicylate separately.

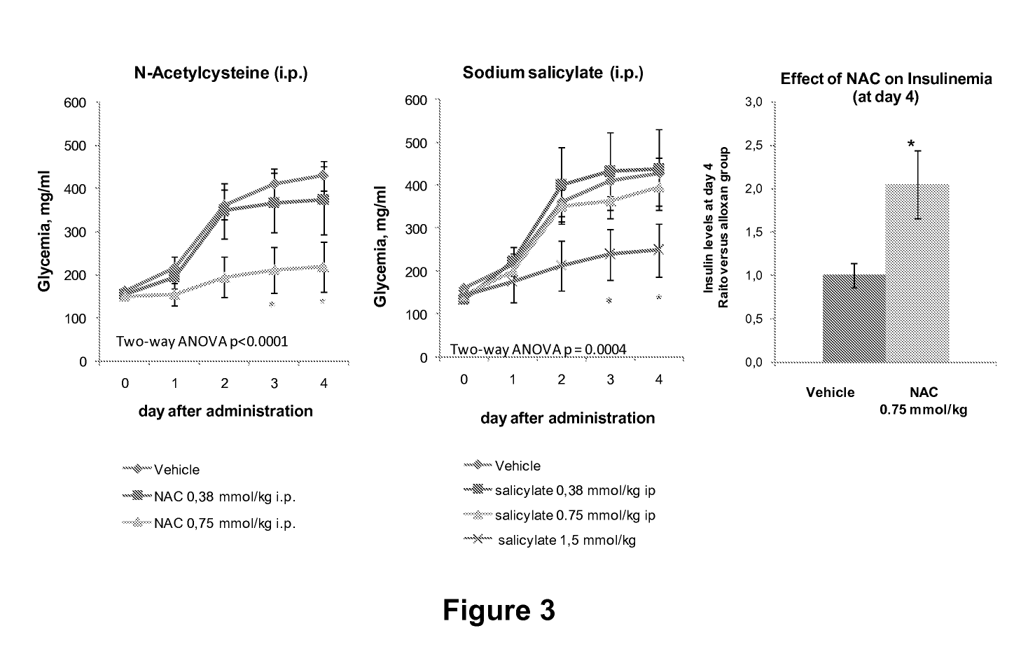

The effects of N-acetylcysteine and sodium salicylate administration on glycemia and insulinemia was assessed by treatment of cd-1 mice with alloxan-induced beta-cell destruction. Salicylate was administered i.p. alone at three different concentrations (0.38, 0.75, and 1.5 mmol/kg) and N-acetylcysteine (NAC) was administered i.p. alone at two different concentrations (0.38 and 0.75 mmol/kg). As shown in FIG. 3, significant reductions of glycemia were observed only at the highest administered concentrations of either compound. Similarly, insulinemia was significantly increased at the highest concentrations of NAC administered to alloxan-treated cd-1 mice.

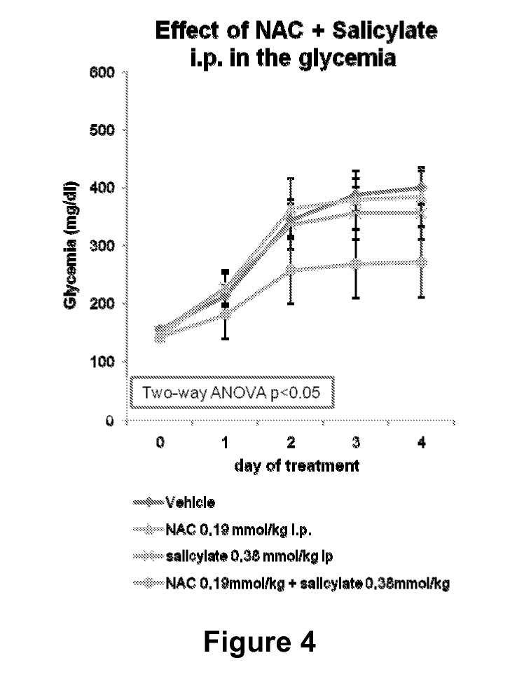

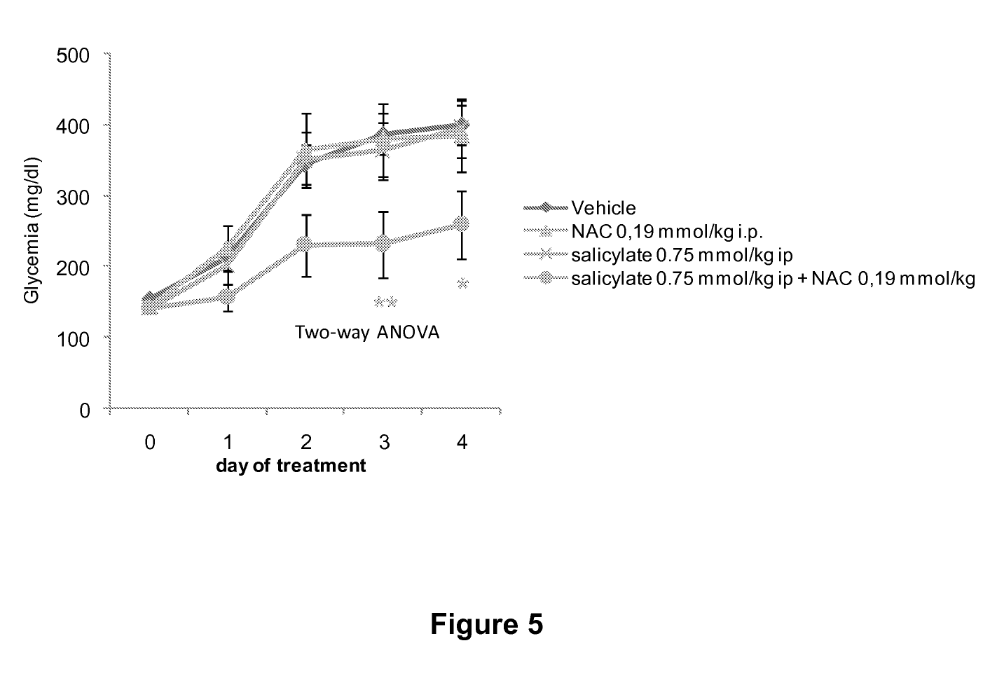

These effects on glycemia after i.p. administration were mimicked at much lower concentrations when NAC and sodium salicylate were administered to alloxan-treated cd-1 mice. Administration of NAC (0.19 mmol/kg) or sodium salicylate (0.38 mmol/kg) in amounts previously shown not to affect glycemia in alloxan-treated cd-1 mice were shown to significantly reduce glycemia when administered together in these amounts. These results are shown in FIG. 4, showing statistical significance in a two-way ANOVA of p<0.05. These results were also found using higher administered salicylate amounts (0.75 mmol/kg), as shown in FIG. 5. Administration of NAC (0.38 mmol/kg) and sodium salicylate (0.75 mmol/kg) to alloxan-treated cd-1 cells at concentrations where both compounds showed statistically-significant reductions in glycemia showed synergistic effects on glycemia having statistical significance of p<0.0001 using two way ANOVA.

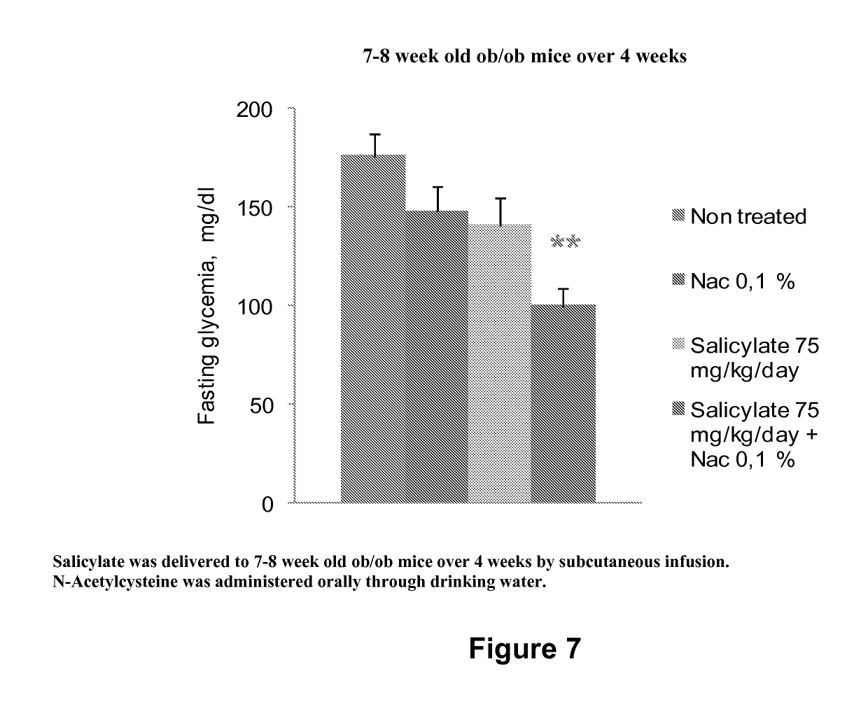

The effects of co-administration of NAC and salicylate were further assessed by chronic administration of these compounds alone or in combination to ob/ob mice. Salicylate (75 mg/kg/day by subcutaneous (s.c.) infusion) and N-acetylcysteine (0.1% drinking water) were administered alone or in combination to 7-week old ob/ob mice for four weeks and the effects on fasting glycemia assessed as set forth herein using a rapid glucose analyzer (Accu-Chek Aviva; Roche). The results of these assays are shown in FIG. 7, wherein administration of the combination of NAC and sodium salicylate was determined to have a statistically-significant reduction in fasting glycemia. Plasma triglycerides and free fatty acids were also determined in these mice, and statistically-significant reductions of free fatty acid and triglyceride levels were found.

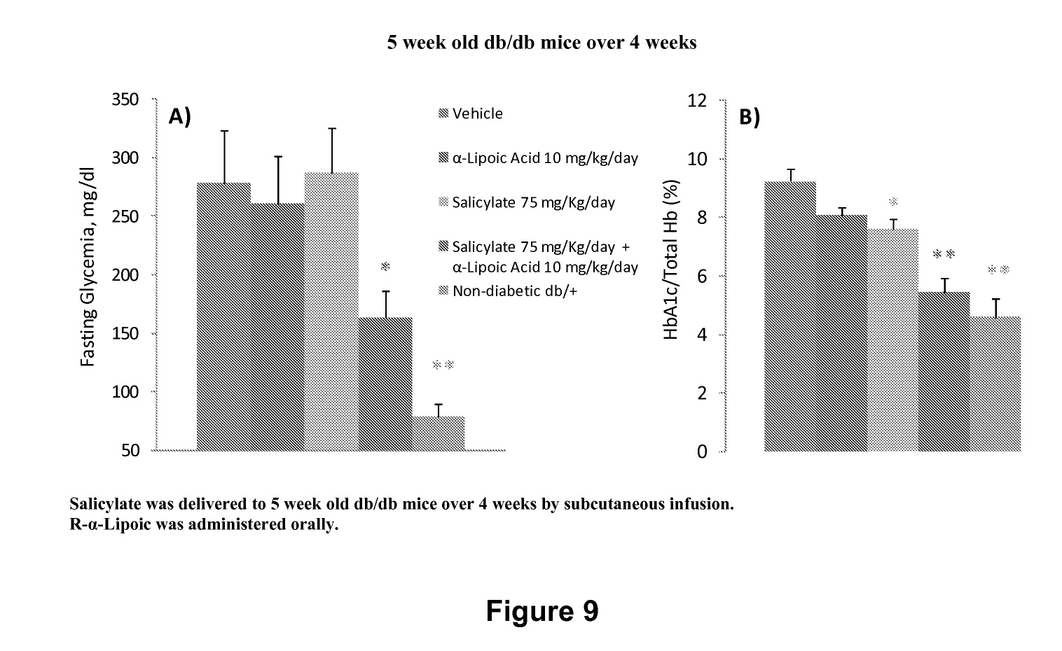

The effects of co-administration of (R) alpha-lipoic acid and salicylate on fasting glycemia in 5 week old db/db mice were determined. (R) alpha-lipoic acid (10 mg/kg/day, administered i.p.) and salicylate (75 mg/kg/day, administered by s.c. infusion) were administered, alone or in combination, over a four-week treatment course and fasting glycemia determined. As shown in FIG. 9, fasting glycemia was reduced by the combination of (R) alpha-lipoic acid and salicylate. The percentage of HbA1c (a measure of long-term glycemia control determined by assessing the extent of glycosylation of red blood cell hemoglobin) to total hemoglobin was determined in these mice after 4 weeks of treatment with (R) alpha-lipoic acid and salicylate alone or in combination under the same conditions and administration amounts of routes used in the glycemia assays. Shown in FIG. 9, combination treatment showed a statistically-significant reduction in HbA1c to levels close to those found in control (db/+) mice.

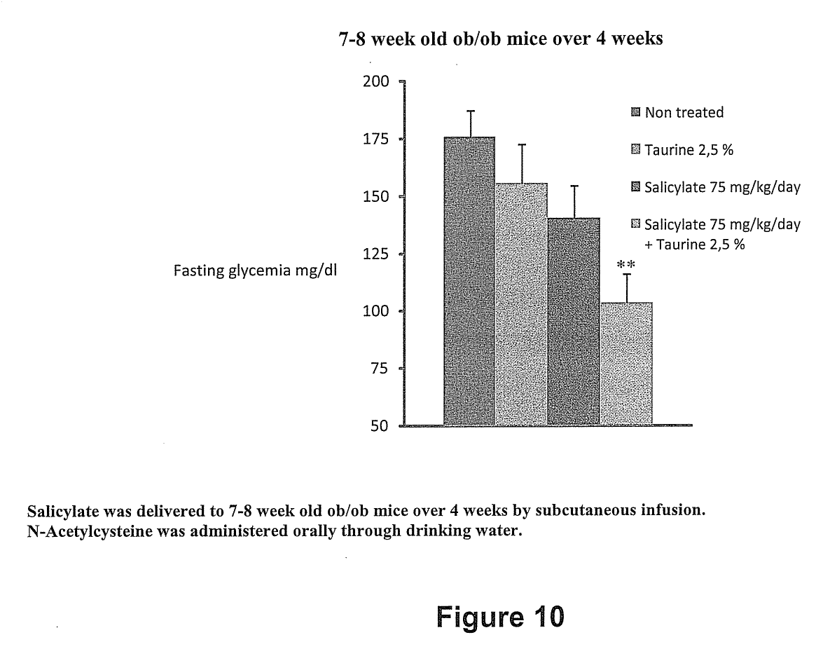

The effects of co-administration of another antioxidant, taurine, and salicylate were assessed by chronic administration of these compounds alone or in combination to ob/ob mice. Salicylate (75 mg/kg/day by subcutaneous (s.c.) infusion) and N-acetylcysteine (2.5% in drinking water) were administered alone or in combination to 7-week old ob/ob mice for four weeks and the effects on fasting glycemia assessed as set forth herein. The results of these assays are shown in FIG. 10, wherein administration of the combination of taurine and sodium salicylate was determined to have a statistically-significant reduction in fasting glycemia.

We claim:

1. A pharmaceutically acceptable composition comprising a therapeutically-effective amount of an antioxidant agent, an anti-inflammatory agent, and at least one pharmaceutically acceptable carrier.

2. The pharmaceutically acceptable composition according to claim 1 wherein the antioxidant agent is resveratrol, silibinin, alpha-lipoic acid or a pharmaceutically acceptable salt thereof, pterostilbene, N-acetyl cysteine, taurine, probucol, idebenone or curcumin.

3. The pharmaceutically acceptable composition according to claim 1 wherein the anti-inflammatory agent is sulindac, salicylic acid, diflunisal, 2-hydroxy-4-trifluoromethylbenzoic acid (HTB), salsalate, naproxen, paracetamol, diclofenac, ibuprofen, dexibuprofen or dexketoprofen.

4. The pharmaceutically acceptable composition according to claim 1 wherein the antioxidant is N-acetyl cysteine, alpha-lipoic acid or a pharmaceutically acceptable salt thereof or taurine and the anti-inflammatory agent is sulindac, salicylic acid, diflunisal, 2-hydroxy-4-trifluoromethylbenzoic acid (HTB), salsalate, naproxen, paracetamol, diclofenac, ibuprofen, dexibuprofen or dexketoprofen.

5. A method of treating a metabolic disorder in a patient comprising administering to the patient in need of such treatment a therapeutically effective amount, particularly a synergistically effective amount of a pharmaceutical composition of a pharmaceutical combination comprising an antioxidant agent and an anti-inflammatory agent.

6. The method according to claim 5 wherein the pharmaceutical composition further comprises at least one other anti-diabetic agent.

7. The method according to claim 5 wherein the pharmaceutical combination comprises an antioxidant agent that is resveratrol, silibinin, alpha-lipoic acid or a pharmaceutically acceptable salt thereof, pterostilbene, N-acetyl cysteine, taurine, probucol, idebenone or curcumin.

8. The method according to claim 5 wherein the pharmaceutical combination comprises an anti-inflammatory agent that is sulindac, salicylic acid, diflunisal, 2-hydroxy-4-trifluoromethylbenzoic acid (HTB), salsalate, naproxen, paracetamol, diclofenac, ibuprofen, dexibuprofen or dexketoprofen.

9. The method according to claim 5 wherein the pharmaceutical composition comprises an antioxidant agent that is N-acetyl cysteine, alpha-lipoic acid or a pharmaceutically acceptable salt thereof or taurine and an anti-inflammatory agent that is sulindac, salicylic acid, diflunisal, 2-hydroxy-4-trifluoromethylbenzoic acid (HTB), salsalate, naproxen, paracetamol, diclofenac, ibuprofen, dexibuprofen or dexketoprofen.

10. The method according to claim 5 wherein the metabolic disorder is Type I diabetes.

11. The method according to claim 5 wherein the metabolic disorder is Type II diabetes.

12. The method according to claim 5 wherein the metabolic disorder is hyperglycemia.

13. The method according to claim 5 wherein the metabolic disorder is insulin resistance.

14. The method according to claim 5 wherein the metabolic disorder is pancreatic β-cell.

15. The method according to claim 5 wherein the metabolic disorder is Latent Autoimmune Diabetes of Adulthood (LADA).

16. The method according to claim 5 wherein the metabolic disorder is dyslipidemia.

17. The method according to claim 5 wherein the metabolic disorder is metabolic syndrome.

18. A method of treating chronic obstructive pulmonary disease in a patient comprising administering to the patient in need of such treatment a therapeutically effective amount, particularly a synergistically effective amount of a pharmaceutical composition of a pharmaceutical combination comprising an antioxidant agent, an anti-inflammatory agent.
